(19) United States
(12) Patent Application Publication
(10) Pub. No.: US 2011/0189257 A1
(43) Pub. Date: Aug. 4, 2011

(54) NANOPARTICLE BASED THERAPY FOR AGGREGATING MUCIN

(75) Inventors: Wei-Chun Chin, Merced, CA (US); Eric Yi-Tong Chen, Merced, CA (US)

(73) Assignee: The Regents of the University of California

(21) Appl. No.: 13/019,921

(22) Filed: Feb. 2, 2011

Related U.S. Application Data
(60) Provisional application No. 61/301,077, filed on Feb. 3, 2010.

(51) Int. Cl. A61F 6/08; A61K 31/722; A61K 31/785; A61K 31/131; A61K 31/4409; A61K 31/14; A61K 31/575; A61K 31/23; A61K 31/255; A61K 38/16; C07K 14/435; A61P 15/18; A61K 9/14; B82Y 5/00

(52) U.S. Cl. 424/430; 514/55; 424/78.36; 514/663; 514/352; 514/642; 514/643; 514/171; 514/552; 514/517; 514/2.3; 530/395; 424/400; 977/915; 977/773

(57) ABSTRACT

There are provided compositions and methods to aggregate mucus using a plurality of positively charged nanoparticles. There are also provided compositions and methods for inducing contraception in the subject.

### CROSS-REFERENCE TO RELATED APPLICATION

This application claims the benefit under 35 U.S.C. §119(e) of U.S. Provisional Ser. No. 61/301,077 filed Feb. 3, 2010, the content of which is incorporated by reference in its entirety into the present disclosure.

### STATEMENT OF GOVERNMENT SUPPORT

This invention was made with government support under Contract No. 1R15HL095039 awarded by the National Institutes of Health (NIH) and Contract No. CBET-0932404 awarded by National Science Foundation (NSF). The government has certain rights to the invention.

### FIELD OF THE INVENTION

This invention relates to compositions and methods for aggregating mucus using positively charged nanoparticles. There are also provided compositions and methods for inducing contraception in the subject.

### BACKGROUND

Birth control is a regimen of one or more actions, devices, sexual practices, or medications followed in order to deliberately prevent or reduce the likelihood of pregnancy or childbirth. One of the routes to prevent or end pregnancy is the prevention of fertilization of the egg by sperm cells (contraception). The fertilization of the egg with the sperm is facilitated by mucus in vaginal tract (cervical mucus). Mucins are a family of glycoproteins that provide the viscoelastic properties to mucus. Mucins are typically secreted from epithelial cells at body surfaces, including the eyes, pancreatic ducts, gallbladder, prostate, respiratory, gastrointestinal and female reproductive tracts. The ability to control the dispersion of mucin or the mucus may provide an alternative route to contraception in a subject.

Traditionally, the contraceptive methods involving vaginal insertion of contraceptive devices can be labor intensive and quite costly. Contraceptive pills, although, are common, result in adverse side-effects and can lead to significant hormonal disregulation. Other methods can have a prolonged incubation time before protection mechanism takes place. Placement of a sperm barrier in vaginal tract has also been shown to cause secondary infections.

Consequently, there is a need to provide compositions and methods for inducing more direct and inexpensive contraception in the subjects.

### SUMMARY

One of the physiological means of controlling birth is by regulating the rheological properties of mucus in vaginal tract (cervical mucus). Hydration and proper dispersion of cervical mucus plays a critical role in regulating fertilization of sperm and oocyte. The aggregation of vaginal/cervical mucus hampers the penetration and fertilization of an egg or an oocyte with a sperm since the thickening of the mucus restricts the mobility of the sperm thereby hampering its penetration. This aggregation of the mucus by the positively charged nanoparticles provides an effective method of contraception in subjects.

Mucins, that are present in the mucus, are typically polyanionic in charge due to their highly glycosylated nature. Without being limited by any theory, the inventors have found that the nanoparticles that have overall positive charge, such as, but not limited to, positively charged amine based (—NH₂) polystyrene nanoparticles favor electrostatic binding with anionic mucin. The electrostatic attraction enables positively-charged nanoparticles to crosslink polyanionic mucin polymer strands to gelate mucin network, thicken mucus and change mucus's rheological properties. The inventors have, therefore, unexpectedly found that the positively-charged nanoparticles serve as a contraceptive material or can be made into a device to gelate vaginal/cervical mucus thereby hampering the sperm penetration through mucus and inhibiting fertilization of the egg with the sperm. Additionally, the presence of positively charged nanoparticles in the vaginal tract of the female can thicken the mucus of the seminal fluid of the male further reducing the penetration of the sperm and inducing contraception.

Disclosed herein are compositions and methods using positively charged nanoparticles for an aggregation of mucus in subjects. The compositions and methods of nanoparticles provided herein avoid the use of conventional hormonal contraceptives, such as, pills and intrauterine devices, that are expensive, invasive, and have numerous side effects. The compositions and methods provided herein for contraception have various advantages, such as, but are not limited to, economical manufacture with commercialization potential; a rapid protective mechanism as the mucin network can be quickly thickened via cross-linking; enable immediate protective response upon application before ejaculation; a high level of stability; ease of modification based on the subject and the body surface; ease of delivery, for example, nanoparticles can be made into ointment/gel for smearing or simple spray-on application; no disregulation of hormonal cycles; and/or reduced side-effects, complications and infections.

In one aspect, there is provided a method to aggregate mucus, comprising, or alternatively consisting essentially of, or yet further consisting of, contacting the mucus with an effective amount of a plurality of positively charged nanoparticle, thereby aggregating the mucus.

Mucin in a subject is aggregated or gelated with a cross-linking network of positively charged ions, such as, but not limited to, potassium, calcium or magnesium ions. For example, Ca²⁺ ions or Mg²⁺ ions cross-link with the negatively charged polyglcosylated mucin by electrostatic attraction resulting in the aggregation or gelation of mucin. This aggregation, however, is reversible and does not thicken the mucus enough to prevent penetration of the egg with the sperm. Without being bound by theory, it is proposed that the contacting of the mucin with a composition comprising an effective amount of the plurality of positively charged nanoparticles causes the replacement of the positively charged ions on mucin (such as Ca²⁺ ions, K⁺ ions, or Mg²⁺ ions) with the positively charged nanoparticles thereby causing the aggregation of mucin and hence, the aggregation of mucus. Applicants have unexpectedly found that the aggregation of mucus with positively charged nanoparticles is irreversible. The positively charged nanoparticles promote mucus to form larger gels which become poorly dispersed and less transportable. This aggregation of the mucus with positively charged nanoparticles prevents the penetration and fertilization of the egg by the sperm leading to contraception.

In one aspect, there is provided a method of contraception in a female mammal, comprising, or alternatively consisting essentially of, or yet further consisting of, administering to the female mammal an effective amount of a plurality of positively charged nanoparticles, resulting in the contraception in the mammal. In some embodiments, the effective amount of the plurality of positively charged nanoparticles causes an aggregation of the mucus thereby causing contraception in the mammal. In yet another aspect, there is provided a method to prevent fertilization of an egg by a sperm by aggregating cervical mucus in a female mammal, comprising, or alternatively consisting essentially of, or yet further consisting of, administering to the female mammal prior to insemination an effective amount of a plurality of positively charged nanoparticle to cause the aggregation of the cervical mucus in the female mammal, resulting in the prevention of the fertilization of the egg by the sperm in the female mammal.

In one aspect, there is provided a composition comprising, or alternatively consisting essentially of, or yet further consisting of, a plurality of nanoparticle, wherein the average diameter of the nanoparticles is less than about 500 nm. In one aspect the plurality of nanoparticles have an overall positive charge. In some embodiments, the nanoparticles comprise polystyrene nanoparticles that are positively charged.

The compositions can be topically applied in the methods of the invention or alternatively, administered by injection, diaphragm, condom, sponge, tampon, suppository, barrier, intrauterine device, or vaginal ring. By varying the size of the nanoparticle, differential effects in reaching different depth of penetration and causing mucus aggregation within the affected tissue or organ can be achieved.

In another aspect, there is provided an in vitro method to aggregate mucus, comprising, or alternatively consisting essentially of, or yet further consisting of, contacting the mucus with an effective amount of a plurality of positively charged nanoparticles, thereby aggregating the mucus.

In another aspect, there is provided a kit for contraception comprising, or alternatively consisting essentially of, or yet further consisting of, a plurality of positively charged nanoparticles; and instructions for use. In another aspect, there is provided a kit for contraception comprising, or alternatively consisting essentially of, or yet further consisting of, a device containing a plurality of positively charged nanoparticles; and instructions for use.

In another aspect, there is provided a device for contraception in a mammal, wherein the device comprises, or alternatively consists essentially of, or yet further consists of, a means for administering an effective amount of a plurality of positively charged nanoparticles for aggregating mucus in a mammal, thereby causing contraception in the mammal.

In another aspect, there is provided an isolated complex of nanoparticle with mucin wherein the nanoparticle is positively charged before complexation with mucin.

In other aspects, there is provided use of the plurality of the positively charged nanoparticles and/or the compositions provided herein in the preparation of a medicament to aggregate mucus in a subject. In yet other aspects, there are provided use of the plurality of the positively charged nanoparticles and/or compositions provided herein in the preparation of a medicament to aggregate mucus to cause contraception in a subject. In yet other aspects, there are provided use of the plurality of the positively charged nanoparticles and/or the compositions provided herein to aggregate mucus to cause contraception in a subject.

### BRIEF DESCRIPTION OF THE DRAWINGS

FIG. 1A illustrates that positively-charged NPs induce mucin aggregation. Various concentrations (solid circles: 100 μg/L, solid triangles: 1 mg/L, solid squares: 10 mg/L) of positively-charged NPs (160 nm) were added to mucin solution (1 mg/L). Significant mucin aggregates were found at 10 mg/L and 1 mg/L of positive NPs. The size of mucin-NPs aggregates were determined with DLS, as described herein.

FIG. 1B illustrates that negatively-charged NPs can not induce mucin aggregation. Various concentrations (solid circles: 100 μg/L, solid triangles: 1 mg/L, solid squares: 10 mg/L) of negatively-charged NPs (120 nm) were added to mucin solution (1 mg/L). Mucin aggregate size remained unchanged (below 300 nm) throughout 72 hrs with three concentrations of NPs. The size of mucin-NPs aggregates were determined with DLS.

FIG. 1C illustrates that neutral NPs can not induce mucin aggregation. Various concentrations (solid circles: 100 μg/L, solid triangles: 1 mg/L, solid squares: 10 mg/L) of neutral NPs (99 nm) were added to mucin solution (1 mg/L). Mucin aggregate size remained unchanged (below 300 nm) throughout 72 hrs with three concentrations of NPs. The size of mucin-NPs aggregates were determined with DLS.

FIG. 1D illustrates that NPs alone can not generate large aggregates. Various concentrations (solid triangles: 1 mg/L solid squares: 10 mg/L) of positively-charged NPs (160 nm) were added to HBSS. The size of aggregates were determined with DLS.

FIG. 1E illustrates that positive NPs induce significant mucin aggregation. Various concentrations (solid triangles: 1 mg/L, solid squares: 10 mg/L) of positively-charged NPs (57 nm) were added to mucin solution (1 mg/L). Significant mucin aggregates were found at 10 mg/L and 1 mg/L of positive NPs. The size of mucin-NPs aggregates were determined with DLS.

FIG. 1F illustrates that NPs alone can not generate large aggregates. Various concentrations (solid triangles: 1 mg/L, solid squares: 10 mg/L) of positively-charged NPs (57 nm) were HBSS. The size of aggregates were determined with DLS.

FIG. 1G illustrates that mucin alone can not generate large aggregates. Mucin solution (1 mg/L) alone failed to generate significant size of aggregates after 72 hrs. The size of aggregates were determined with DLS.

FIG. 2 illustrates SEM micrographs of mucin-NPs complexes with 160 nm (A, C) and 57 nm (B, D) positively-charged NPs after 5 hrs (A, B) and 72 hrs (C, D). There SEM images clearly demonstrate that NPs and mucins aggregate together form large mucin-NPs gels. The black holes in the images are the pores in Isopore membranes. The scale bar is 1 μm.

FIG. 3A illustrates that EGTA (Ca²⁺ chelator) can not disperse NPs-induced mucin aggregates. Positively-charged NPs (160 nm, 10 mg/L) induced mucin (1 mg/L) to aggregate forming large size mucin-NPs aggregates (˜6 μm) in 72 hrs. EGTA (2 mM) was added to chelate Ca²⁺ ions that can cross-link mucins forming gels. However, EGTA could not disperse mucin-NPs aggregates after 72 hrs incubation.

FIG. 3B illustrates that EGTA (Ca²⁺ chelator) can not disperse NPs-induced mucin aggregates. Positively-charged NPs (57 nm, 10 mg/L) induced mucin (1 mg/L) to aggregate forming large size mucin-NPs aggregates (˜6 μm) in 72 hrs. EGTA (2 mM) was added to chelate Ca²⁺ ions that can cross-link mucins forming gels. However, EGTA could not disperse mucin-NPs aggregates after 72 hrs incubation.

FIG. 4A-B show Effects of NPs on swelling kinetics and diffusivity of secretory granule matrix.

(A) Data points of swelling kinetics of secretory granule matrices of A549 cells were fit with equation (1). Exocytosis was triggered by ionomycin. Two representative lines are displayed here. The control (NP free; open circles) shows a higher rate of swelling of newly released mucin network than the swelling rate of mucin matrix when exposed to positively-charged NP (160 nm, 1 mg/L, solid circle). The black arrows indicate the swelling of exocytosed mucin matrix at different time points. (B) Positively-charged NPs hinder the rate of mucin diffusivity (hydration). Mucin post-exocytotic swelling from A549 cells was significantly reduced by positively-charged NPs (160 and 57 nm). This protocol to estimate mucin diffusivity of newly released mucin gels from A549 cells was adapted from the inventors' previous study (Chin et al. Macromolecular Symposia (2005) 227(1):89-96). Data are shown as mean±SD (n>10). NP treated groups are significantly different from the untreated control at p<0.005 as indicated by *.

### DETAILED DESCRIPTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); Current Protocols in Immunology (J. E. Coligan, et. al. eds., (1997)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; and Animal Cell Culture (R. I. Freshney, ed. (1987)).

It is to be understood, although not always explicitly stated that all numerical designations, e.g., pH, temperature, time, concentration, and molecular weight, including ranges, are approximations which are varied (+) or (−) by increments of 1.0 or 0.1, as appropriate. The term “about” includes the exact value “X” in addition to minor increments of “X” such as “X+1” or “X 1” and deviation of ±15%, or alternatively ±10%, or alternatively ±5%. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a nanoparticle” includes a plurality of nanoparticles, including mixtures thereof.

An “administration” refers to the delivery of a medication, such as a nanoparticle, a plurality of nanoparticles, and/or composition of nanoparticles of the invention to an appropriate location of the subject in vivo or in vitro, where a desired effect is achieved. Non-limiting examples include topical, via injection, diaphragm, condom, tampon, suppository, sponge, barrier, intrauterine device, vaginal ring, or the like. Various physical and/or mechanical technologies are available to permit the sustained or immediate topical or transdermal administration of nanoparticles.

An “aggregation” or “aggregating,” refers to gelation of mucin or mucus to form large gels. The gelation of mucus results in enhanced viscosity of mucus as compared to an un-gelated mucus or mucus normally gelated with positively charged ions such as, calcium, potassium, or magnesium.

A “composition” is intended to include the combination of an active agent with a carrier, inert or active such as saline or water, making the composition suitable for diagnostic or therapeutic use in vitro, in vivo or ex vivo.

“Comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

An “effective amount” or a “therapeutically effective amount” is an amount sufficient to effect beneficial or desired results. An effective amount can be administered in one or more administrations, applications or dosages.

An “egg,” “oocyte,” “ovocyte,” or “ocyte,” refers to a female gametocyte or germ cell involved in reproduction. It is an immature ovum, or egg cell.

“Mucin” or “mucins,” as used herein, refers to the glycol-peptides of mucus secreted from epithelial cells that form mucosal barrier to protect various tissues, such as the eyes, pancreas, intestine, exocrine glands, hepatobiliary, respiratory and reproductive tracts. There are approximately 20 different types of mucins known in the art, e.g. MUC 1, MUC 2, MUC 5AC and MUC 5B . . . etc. Typically, mucins form extremely large oligomers through linkage of glycoprotein monomers using disulfide bonds. Usually, such glycoproteins are large >100,000 daltons and typically consist of approximately 75% carbohydrate and 25% protein. Altered mucins, which contain abnormal concentration of sulfate, sialic acid or fucose, also occur in pathological conditions, such as inflammatory diseases.

“Mucus” as used herein, refers to the mixtures of different types of mucins, actins, DNA or other glycol-proteins that form the hydrated layer on the surface of various tissues, such as the reproductive tracts.

“Nanoparticle” as used herein denotes a structure which is biocompatible with and sufficiently resistant to chemical and/or physical destruction by the environment of use such that a sufficient amount of the nanoparticles remain substantially intact after delivery to the site of application or treatment or incubated with an in vitro sample so as to be able to reach mucus or reach the nucleus of a cell or some other cellular structure. The nanoparticles may undergo biodegradation upon aggregation of the mucus or upon entry of a cell's nucleus.

The term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)). The term includes carriers that facilitate controlled release of the active agent as well as immediate release.

The term “plurality” refers to more than one nanoparticle. The term “plurality” and “one or more” are used interchangeably herein.

“Topical administration” refers to delivery of a medication by application to the mucosal membrane or skin Non-limiting examples of topical administration include any methods described under the definition of “administration” pertaining to delivery of a medication to appropriate area. For topical use, the pharmaceutically acceptable carrier is suitable for manufacture of, creams, ointments, jellies, gels, solutions, suspensions, etc. Such carriers are conventional in the art. These formulations may optionally comprise additional pharmaceutically acceptable ingredients such as diluents, stabilizers, and/or adjuvants.

A “mammal” for treatment includes a human. Non-human mammals subject to treatment include, for example conventional animal models such as murine, such as rats, mice, canine, such as dogs, leporids, such as rabbits, bovine, ovine, feline, and equine.

### Nanoparticles and Compositions

In one aspect, there is provided a composition comprising, or alternatively consisting essentially of, or yet further consisting of, one or more of nanoparticles which have an overall positive charge. In some embodiments, the average diameter of the nanoparticles is less than about 500 nm. The composition of the invention is used in the methods as described herein. In some embodiments, the nanoparticle is a polystyrene nanoparticle that is positively charged. Drugs, bioactive or other relevant materials can be incubated with the nanoparticles, and thereby be adsorbed or attached to the nanoparticle.

In some embodiments, the aggregation of mucus increases with a decrease in the size of the positively charged nanoparticle contacted with it. Nanoparticles can be solid colloidal particles ranging in size from 1 to 1000 nm. Nanoparticle can have any diameter less than or equal to 5 nm, or alternatively less than about 10 nm, or alternatively less than about 15 nm, or alternatively less than about 20 nm, or alternatively less than about 25 nm, or alternatively less than about 30 nm, or alternatively less than about 50 nm, or alternatively less than about 100 nm, or alternatively less than about 150 nm, or alternatively less than about 200 nm, or alternatively less than about 300 nm, or alternatively less than about 400 nm, or alternatively less than about 500 nm. In some embodiments, the nanoparticle has a diameter between about 100 nm to about 500 nm; or alternatively between about 50 nm to about 200 nm; or alternatively between about 150 nm to about 300 nm; or alternatively between about 200 nm to 500 nm; or alternatively between about 100 nm to about 150 nm; alternatively between about 1 nm to about 50 nm; alternatively between about 1 nm to about 100 nm; alternatively between about 50 nm to about 100 nm; alternatively between about 150 nm to about 200 nm; alternatively between about 200 nm to about 300 nm; alternatively between about 300 nm to about 400 nm; or alternatively between about 400 nm to about 500 nm.

In some embodiments, the mucus, in addition to being contacted with the plurality of the positively charged nanoparticles, is further contacted concomitantly or sequentially with a spermicide. In one aspect, the spermicide is administered prior to the nanoparticles. In another aspect, the spermicide is administered after the nanoparticles. A wide variety of spermicides may be used along with the composition of the invention to kill, immobilize or otherwise render sperm cells inactive in the vagina. Any compatible, water-soluble spermicide may be used in the composition of the invention. The examples of spermicide include, but are not limited to, detergent nonoxynol-9 (N-9, nonylphenoxypolyethoxyethanol), benzalkonium chloride, sodium cholate, p-diisobutylphenoxypolyethoxyethanol (octoxynol), p-methanylphenyl polyoxyethylene (8.8) ether (menfegol), dodecamethylene glycol monolaurate, sodium lauryl sulfate, and gramicidin.

The amount of spermicide contained in the composition or the devices containing the composition of the invention vary in accordance with their rate of release from the device and their spermicidal efficacy.

In some embodiments, the composition comprising the positively charged nanoparticle optionally containing a spermicide further comprise a therapeutic drug useful for the prevention or treatment of sexually transmitted diseases. Alternatively, the composition is administered concurrently or simultaneously with the therapeutic drug. The examples of the therapeutic drug useful for the prevention or treatment of sexually transmitted diseases include, but are not limited to, vaginal anti-fungals, anti-HIV, metronidazole, and miconazole nitrate.

In some embodiments, the compositions provided herein further comprise a biodegradable dendrimer. A biodegradable dendrimer with multiple side-groups can further enhance contraceptive efficacy in crosslinking mucin. Dendrimers are highly branched polymers with a three-dimensional structure. These monodisperse molecules are composed of an initiator core, interior layers of repeating units, and multitudinous terminal groups. They are classified by the number of branches and terminal groups. Various cores and units can be used, which can change the properties and shape of the dendrimer. The examples of dendrimers include, but are not limited to, polylysine, polyamidoamine (PAMAM), poly(etherhydroxylamine) (PEHAM) or polyporpyleneimine dendrimer scaffold. Because of its multiple terminal groups and its polymer backbone, dendrimers can have multiple functionalities. Dendrimers can be used for synthesizing hydrogels, cross-linked networks that increase in volume in aqueous solution. By adding polyethylene glycol or PEG groups to the dendrimers, these hydrogels can be used to attach to mucin.

The present invention encompasses devices whose construction allows them to be positioned within the vagina such that they substantially block the access of the sperm to the cervix and provide controlled release of the composition provided herein, by diffusion from the device.

There are a number of advantages of the present invention. One advantage of the invention is the ability to use nanoparticles that comprise a material that is biologically inert and can be physiologically tolerated without significant adverse effects by biological systems. Further, a nanoparticle can be comprised of a biodegradable material. There are no limits on the physical parameters of a nanoparticle component of the present invention, although the design of a delivery vehicle should take into account the biocompatibility of the nanoparticle vehicle and the effect on the overall charge. The physical parameters of a nanoparticle vehicle can be optimized, with the desired effect governing the choice of size and shape. For example, the nanoparticle sizes for transport to a cell's nucleus can be on the order of 5 nm where larger particles would be desired for a given application. Additionally, particles smaller than about 25 nm in diameter can be used in nuclear targeting to facilitate entry into the nucleus via a nuclear pore.

The positively charged nanoparticle can comprise a variety of materials including, but not limited to, polymers such as polystyrene, silicone rubber, polycarbonate, polyurethanes, polypropylenes, polymethylmethacrylate, polyvinyl chloride, polyesters, polyethers, and polyethylene. Biodegradable, biopolymer (e.g. polypeptides such as BSA, polysaccharides, etc.), other biological materials (e.g. carbohydrates), and/or polymeric compounds are also suitable for use as a nanoparticle scaffold. The invention encompasses any nanoparticle that is positively charged. The nanoparticles may themselves have a positive charge or may be modified to attach a positive charge to the scaffold, such as, but not limited to, amine, chitosan, polyethyleneimine (PEI), stearyl amine, long polymer connected to amine group, 4-(dimethylamino) pyridine gold nanoparticle. Factors such as nanoparticle surface charge and hydrophilic/hydrophobic balance of these polymeric materials can be achieved by synthetic modification of the polymers. Such synthetic modification is well within the skill of the skilled artisan, examples of which are described below. Various methods for producing the positively charged nanoparticles are described in Klesing et al. (2009) J. Mater Sci.: Mater Med. (available at the web address www.springerlink com/content/jk581q0420800271/); Tan et al. (2007) Langmuir 23(19):9836-9843; Hong et al. (2009) Chem. Eur. J. 15:5935-5941; Yiu et al. (2009) J. Biomed Mater. Res. Part A:386-392; and Yu et al. (2009) J. Mater. Chem. 19:8928-8935.

Examples of positively charged nanoparticles include, but are not limited to, those described in the preceding paragraph, for example poly(ethyleneimine) (PEI)-coated nanoparticles (Klesing et al. (2009) J. Mater Sci.: Mater Med. (available at the web address www.springerlink com/content/jk581q0420800271/); gold, silver and platinum-coated nanoparticles (Hong et al. (2009) Chem. Eur. J. 15:5935-5941); polyethyleimine-coated Fe₃O_{4—}MCM-48 nanocomposite particles (Yiu et al. (2009) J. Biomed Mater. Res. Part A:386-392); titanium dioxide nanotubes with gold nanoparticles (Graham et al (2009); and silver nanoparticles (Tan et al. (2007) Langmuir 23(19):9836-9843). In some embodiments, the surface charge on the nanoparticle is from about 0.1 μeq/g to about 2000 eq/g; about 1 μeq/g to about 1000 eq/g; about 1 μeq/g to about 900 eq/g; about 1 μeq/g to about 800 eq/g; about 1 μeq/g to about 700 eq/g; about 1 μeq/g to about 600 eq/g; about 1 μeq/g to about 500 eq/g; about 1 μeq/g to about 400 eq/g; about 1 eq/g to about 300 eq/g; about 1 μeq/g to about 200 eq/g; about 1 μeq/g to about 100 eq/g; about 0.1 μeq/g to about 1000 eq/g; or about 0.1 μeq/g to about 100 eq/g. In some embodiments, for the amine surface modified polymer, the surface charge ranges from about 1 μeq/g to about 100 eq/g.

The surface charge of the nanoparticle and the zeta potential can be determined using a Malvern Zetamaster. Zeta potential is an abbreviation for electrokinetic potential in colloidal systems. Zeta potential is the potential difference between the dispersion medium and the stationary layer of fluid attached to the dispersed particle. The value of zeta potential can be related to the stability of colloidal dispersions (e.g. a solution of the nanoparticle). The zeta potential indicates the degree of repulsion between adjacent, similarly charged particles in a dispersion. For molecules and particles that are small enough, a high zeta potential will confer stability, i.e. the solution or dispersion will resist aggregation. When the potential is low, attraction exceeds repulsion and the dispersion will break and flocculate. So, colloids with high zeta potential (negative or positive) are electrically stabilized while colloids with low zeta potentials tend to coagulate or flocculate. In some embodiments, the zeta potential of the nanoparticles with the overall average positive charge is in the range of +20 to +40 mV.

Nanoparticles comprising the above materials and having diameters less than 1,000 nanometers are available commercially or they can be produced from progressive nucleation in solution (e.g., by colloid reaction), or by various physical and chemical vapor deposition processes, such as sputter deposition.

In one aspect, there is provide an isolated complex of nanoparticle with mucin wherein the nanoparticle is positively charged before complexation with mucin. The isolated complex of nanoparticle with mucin results in the aggregation of the mucin. The aggregated size of the mucin in the complex is greater than the size of mucin before complexation.

Besides sputter deposition, plasma-assisted chemical vapor deposition (PACVD) is another technique that can be used to prepare suitable nanoparticles. PACVD functions in relatively high atmospheric pressures (on the order of one ton and greater) and is useful for generating particles having diameters of about 1000 nanometers and smaller. The PACVD system typically includes a horizontally mounted quartz tube with associated pumping and gas feed systems. A susceptor is located at the center of the quartz tube and heated using a 60 KHz radio frequency source. The synthesized particles are collected on the walls of the quartz tube. Nitrogen gas is commonly used as the carrier. A constant pressure in the reaction chamber of 10 ton is generally maintained to provide deposition and formation of the ultrafine nanoparticles. PACVD can be used to prepare a variety of suitable biodegradable nanoparticles.

### Therapeutic Utilities

The nanoparticles and/or the compositions provided herein have the following therapeutic utilities.

In one aspect, there is provided a method to aggregate mucus, comprising, or alternatively consisting essentially of, or yet further consisting of, contacting the mucus with an effective amount of a plurality of positively charged nanoparticles, thereby aggregating the mucus.

In some embodiments, the contacting of the mucus with the plurality of the positively charged nanoparticles causes a replacement of calcium ion cross-linking a mucin in the mucus with a positively charged nanoparticle. These methods can be practiced in vitro or in vivo. When practiced in vitro, they provide a suitable cell free system to test or screen for agents or formulations that may augment the inventive compositions as provided herein. To practice the screen, the mucin is prepared in two separate containers and one is contacted with the composition of this invention and the second is contacted with the test agent or composition and the dispersion is compared to the container containing the inventive composition. A third negative control containing the mucin and no agent or formulation may also be included. When practiced in vivo, the methods provide a biological utility as described in more detail below.

In one aspect, there is provided a method of contraception in a female mammal, comprising, or alternatively consisting essentially of, or yet further consisting of, administering to the mammal an effective amount of a plurality of a positively charged nanoparticles, resulting in the contraception in the female mammal. In some embodiments, the effective amount of the plurality of a positively charged nanoparticles causes an aggregation of the mucus causing contraception in the mammal. In another aspect, there is provided a method to prevent fertilization of an egg by a sperm by aggregating cervical mucus in a female mammal, comprising, or alternatively consisting essentially of, or yet further consisting of, administering to the mammal an effective amount of a plurality of a positively charged nanoparticles to cause the aggregation of the cervical mucus in the female mammal, resulting in the prevention of the fertilization of the egg by the sperm in the female mammal.

In some embodiments, the aggregation of mucus by the one or more of the positively charged nanoparticles is irreversible. In some embodiments, the plurality of the positively charged nanoparticles present in the cervical mucus also causes an aggregation of the sperm mucus after insemination thereby further reducing the mobility of the sperm and causing contraception.

The present methods can be practiced on a variety of mammals in which contraception or the prevention of the fertilization of the egg by the sperm is desired. Mammals that can be treated by these methods include animals such as porcine, livestock, and mice as well as human patients. The invention is not to be so limited to any particular amount of the aggregation of the mucus so long as the gelation and the viscosity of the mucous has risen to a degree that there is a prevention of the fertilization of the egg with the sperm.

In some embodiments, the plurality of the positively charged nanoparticles in the methods provided herein, result in an increase in a size of the mucin or the mucus aggregate. In some embodiments, the aggregation of the mucus results in an increase in a size of the mucus to more than about 200 nm in less than about 1 hr of the contacting or the administration of the composition provided herein. In some embodiments, the increase in the size of the mucus is more than about 200 nm; alternatively more than about 300 nm; alternatively more than about 400 nm; alternatively more than about 500 nm; alternatively more than about 600 nm; alternatively more than about 700 nm; alternatively more than about 800 nm; alternatively more than about 900 nm; alternatively more than about 1000 nm; alternatively more than about 2000 nm; alternatively more than about 3000 nm; alternatively more than about 4000 nm; alternatively more than about 5000 nm; alternatively more than about 6000 nm; alternatively more than about 7000 nm; alternatively more than about 8000 nm; alternatively more than about 9000 nm; alternatively more than about 10,000 nm; alternatively more than about 1 μm; alternatively more than about 10 μm; alternatively more than about 20 μm; alternatively more than about 30 μm; alternatively more than about 40 μm; alternatively more than about 50 μm; alternatively more than about 60 μm; alternatively more than about 70 μm; alternatively more than about 80 μm; alternatively more than about 90 μm; alternatively more than about 100 μm; alternatively more than about 200 μm; alternatively more than about 300 μm; alternatively more than about 400 μm; alternatively more than about 500 μm; alternatively more than about 600 μm; alternatively more than about 700 μm; alternatively more than about 800 μm; alternatively more than about 900 μm; or alternatively more than about 1000 μm.

In some embodiments, the increase in the size of the mucus is between more than about 200 nm to about 100 μm; alternatively between more than about 200 nm to about 75 μm; alternatively between more than about 200 nm to about 50 μm; alternatively between more than about 200 nm to about 25 μm; alternatively between more than about 200 nm to about 10 μm; alternatively between more than about 200 nm to about 1 μm; alternatively between more than about 200 nm to about 900 nm; alternatively between more than about 200 nm to about 800 nm; alternatively between more than about 200 nm to about 700 nm; alternatively between more than about 200 nm to about 600 nm; alternatively between more than about 200 nm to about 500 nm; alternatively between more than about 200 nm to about 400 nm; alternatively between more than about 200 nm to about 300 nm.

In some embodiments, the above recited aggregation of the mucus is in less than about 72 hr; alternatively less than about 48 hr; alternatively less than about 24 hr; alternatively less than about 10 hr; alternatively less than about 8 hr; alternatively less than about 5 hr; or alternatively less than about 1 hr of the contacting or the administration of the composition provided herein.

In some embodiments, the aggregation of the mucus in the methods provided herein, results in an increase in viscosity of the mucus, e.g. an increase of 5%, or alternatively 10%, or alternatively 15%, or alternatively 20%, or alternatively 25%, or alternatively 30%, or alternatively 35%, or alternatively 40%, or alternatively 45%, or alternatively 50%, or alternatively 55%, or alternatively 60%, or alternatively 65%, or alternatively 70%.

### Pharmaceutical Formulations and Dosages

In some embodiments, the composition further comprises, or alternatively consists essentially of, or yet further consists of a pharmaceutically acceptable carrier. In another aspect, the compositions contain carriers that modulate (controlled release) the release of the nanoparticle when administered to a subject in need thereof. In a further aspect, the compositions are suitable for topical application to the mucosal surface of a subject in need of such treatment.

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The compositions of this invention are formulated for pharmaceutical administration to a mammal, preferably a human being or livestock. Such pharmaceutical compositions of the invention may be administered in a variety of ways, preferably topically.

In some embodiments, the composition containing the nanoparticles of this invention is administered by contacting the composition with the mucous or mucous-producing cells in the vaginal tract. The compositions are administered in a manner compatible with the dosage formulation, and in a therapeutically effective amount. The quantity to be administered and administration regimen depends on the subject to be treated such as the age, body weight, general health, sex and diet, renal and hepatic function of the subject, capacity of the subject's system to utilize the active ingredient, the mode of administration, the time of administration, rate of excretion, drug combination, and time period for which the effect is desired. Precise amounts of active ingredient required to be administered depend on the judgment of the practitioner and are peculiar to each individual. Determination of an effective dosage is well within the capabilities of those skilled in the art. However, suitable dosage ranges for application are disclosed herein and depend on the manner of administration. Suitable regimes for administration are also variable, but are typified by an initial administration followed by repeated doses at few days or one day intervals. Alternatively, continuous delivery of the composition from the intrauterine device loaded with the composition or the gel or the paste containing the composition or the vaginal ring coated with the composition are contemplated.

Thus, the administration of the composition can be in the form of a single unit dose, multiple doses, or during continuous delivery through an implanted device. The term “unit dose” when used in reference to the composition of the present invention refers to physically discrete units suitable as unitary dosage for the subject, each unit containing a predetermined quantity of active positively charged nanoparticles calculated to produce the desired therapeutic effect in association with the required diluent, i.e., carrier, or vehicle.

Initial dosages can be estimated from in vivo data, such as animal models. Animal models useful for testing the efficacy of nanoparticles to induce contraception are known in the art. Suitable animal models include, but are not limited to murine, such as rats, mice, canine, such as dogs, leporids, such as rabbits, bovine, ovine, feline, and equine. Ordinarily skilled artisans can routinely adapt such information to determine dosages suitable for human administration or any other animal.

The amount of nanoparticle that can be administered to the subject will typically be in the range of from about 200 μg/m³ to about 1 μg/m³; or about 150 μg/m³ to about 1 μg/m³; or about 100 μg/m³ to about 1 μg/m³; or about 50 μg/m³ to about 1 μg/m³; or about 10 μg/m³ to about 1 μg/m³. In some embodiments, the dosage of nanoparticles in the solution is in the range from about 100 mg/L to about 10 g/L.

In some embodiments, the amount of the nanoparticles used may vary from about 1-95% based on the total weight of the composition; or alternatively in the range of from about 5-90% by weight; or alternatively in the range of from about 10-50% by weight; or alternatively in the range of from about 10-80% by weight; or alternatively in the range of from about 50-75% by weight; or alternatively in the range of from about 25-75% by weight; or alternatively in the range of from about 10-75% by weight; or alternatively in the range of from about 5-75% by weight; or alternatively in the range of from about 5-50% by weight; or alternatively in the range of from about 1-10% by weight; or alternatively in the range of from about 1-25% by weight; or alternatively in the range of from about 50-60% by weight; or alternatively in the range of from about 75-95% by weight; or alternatively in the range of from about 40-60% by weight, based on the total weight of the composition.

The amount can be higher or lower, depending upon, among other factors, the activity of the nanoparticle, toxicity, device for administration, and various factors discussed above. Dosage amount and interval can be adjusted individually to provide an effective amount of the nanoparticles which are sufficient to maintain the contraceptive effect. For example, the nanoparticles can be administered once per week, several times per week (e.g., every other day), once per day, or multiple times per day. In cases of local administration or selective uptake, such as local topical administration, the effective local concentration of nanoparticles may not be related to plasma concentration. Skilled artisans will be able to optimize effective local amounts without undue experimentation.

Preferably, the agents and/or compositions will provide therapeutic or prophylactic benefit without causing substantial toxicity. Toxicity of the nanoparticles can be determined using standard pharmaceutical procedures. The dose ratio between toxic and therapeutic (or prophylactic) effect is the therapeutic index. The nanoparticles that exhibit high therapeutic indices are preferred. The compositions of the invention are of sufficiently high pharmaceutical quality, i.e., they are pharmaceutically stable over a storage time of some years, preferably at least one year, more preferably two years.

Other pharmacologically acceptable excipients may also be added to the composition according to the invention. By adjuvants and additives are meant, in this context, any pharmacologically acceptable and therapeutically useful substance which is not an active substance, but can be formulated together with the active substance in the pharmacologically suitable solvent, in order to improve the qualities of the active substance formulation. Preferably, these substances have no pharmacological effects or no appreciable or at least no undesirable pharmacological effects in the context of the desired therapy. The adjuvants and additives include, for example, stabilisers, antioxidants and/or preservatives known in the art which prolong the shelf life of the finished pharmaceutical formulation. The additives also include pharmacologically acceptable salts such as sodium chloride, for example. The preferred excipients include antioxidants such as ascorbic acid, vitamin A, vitamin E, tocopherols and similar vitamins or provitamins occurring in the human body.

Preservatives can be added to protect the formulation from contamination with pathogenic bacteria. Suitable preservatives are those known from the prior art, particularly benzalkonium chloride or benzoic acid or benzoates such as sodium benzoate in the concentration known from the prior art. The amount of benzalkonium chloride is between 1 mg and 50 mg per 100 ml of formulation, about 7 to 15 mg per 100 ml, or about 9 to 12 mg per 100 ml of the formulation according to the invention.

In some embodiments, it may be important to ensure homogenous dispersion of the nanoparticles in suspension, without the formation of aggregates. The formation of more or less compact aggregates can give rise to problems of distribution and therefore of uniformity of dose during the filling of the containers.

In some embodiments, the composition according to the invention is sterile. In some embodiments, the composition of the invention is devoid of preservatives and bacteriostatics. This can prevent the induction of the allergic reactions or irritation in the subjects. Various processes can be used to manufacture sterile pharmaceutical formulations. For example, the active ingredient can be sterilized by dry heating or irradiation such as with gamma rays, followed by preparation of the formulation under aseptic conditions, or the formulation can be pre-prepared and sterilized by treatment in an autoclave or by filtration.

The agents and compositions of the present invention can be used in the manufacture of medicaments and for the treatment of humans and other animals by administration in accordance with conventional procedures, such as an active ingredient in pharmaceutical compositions.

### Drugs Screens and Assays

In one aspect, there is provided an in vitro method to aggregate mucus, comprising, or alternatively consisting essentially of, or yet further consisting of, contacting the mucus with an effective amount of a plurality of positively charged nanoparticles, thereby aggregating the mucus. In some embodiments, the size of the nanoparticle is less than about 500 nm. When nanoparticles are being introduced into cells suspended in a cell culture, the cells are incubated together with the nanoparticle in an appropriate growth media, for example Luria broth (LB) or a suitable cell culture medium. When in vitro experiments are to be performed, nanoparticles can be added directly to a selected cell growth medium before cells are introduced into the medium. Such a medium may be compatible not only with the physiological requirements of the cells, but also with the chemical and reactivity profile of the nanoparticles. The nanoparticles's profile will be apparent to one of skill in the art upon review of the present disclosure.

In some embodiments, there is provided an in vitro method to screen for nanoparticles that aggregate mucin or mucus, comprising, or alternatively consisting essentially of, or yet further consisting of (i) administering a candidate nanoparticles to a test sample containing mucin or mucus; (ii) determining the aggregation of the mucin or the mucus in the test sample; (iii) comparing the aggregation of the mucin or the mucus in the test sample with a control cell where the control cell does not have the nanoparticles in the composition, or the control cell has nanoparticles that aggregate mucin or mucus, thereby screening for the candidate nanoparticles that aggregate mucin or mucus. The methods to determine the aggregation of mucin or mucus in a sample include, but are not limited to, dynamic laser scattering that determines the size of the mucin or the mucus before and after aggregation.

In some embodiments of the in vitro methods, a composition comprising a calcium ion or any other suitable positively charged ion may be added to mucin to cause the aggregation of mucin in the sample. The composition of the positively charged nanoparticle, as provided herein, is then added to the aggregated mucin to cause further aggregation of the mucin.

For the in vitro methods, mucins can be derived from a wide variety of “natural” sources including, but not limited to porcines, bovines, goats, sheep, cattle, felines, non-human primates, humans, and the like. Alternatively, mucins can be chemically prepared. Mucins are selected from buccal and gastrointestinal mucins. The term buccal and gastrointestinal mucins are intended to designate any mucin which is present in the oral cavity or in the gastrointestinal system, respectively. Typical examples are mucins from salivary glands and gastric mucins.

The mucin concentration is selected to reflect the mucin concentration of, and hence to achieve a viscosity similar to, the naturally occurring fluid(s) for which the fluid standard stands as a surrogate. Thus, in pathological conditions where fluid viscosity is abnormal, the mucin concentration of the fluid standard will be adjusted to approximate the viscosity of the abnormal fluid to provide a standard for testing methods and devices used in the pathological patient. Conversely, where fluid viscosity is “normal,” mucin concentrations of the fluid standard will be adjusted to reflect normal fluid viscosity.

Mucins or mucus can also be obtained from commercial sources (e.g. Sigma Chemical Co., St. Louis, Mo., USA)) or, as indicated above, isolated directly from various non-human mammals. Methods of isolating mucus are well known to those of skill in the art. For example, porcine gastric mucus is typically obtained as a by-product in the production of pepsin from hog stomachs. The mucus can be additionally purified by multiple alcohol precipitations, such as 2-3 precipitations with 60% ethanol. During the precipitations, and during the manipulation of the mucus, the use of gentle conditions will result in minimizing of viscosity-decreasing degradation.

In the in vitro methods of the invention, the delivery of the nanoparticles to the mucin or the mucus can be detected on both the interior and exterior of cells or the mucin or the mucus in a variety of ways. One method of detecting the presence of a nanoparticles is by monitoring a sample for the homeostatic change the nanoparticle delivery is designed to produce. For some applications, however, it might be desirable to monitor the presence of a nanoparticle delivery by methods including, but are not limited to, the use of transmission electron, fluorescence and other microscopy techniques; spectroscopic-based detection; and detection methods involving proteins, such as immunological methods.

Transmission electron microscopy (TEM) can be used to determine the presence of a nanoparticles. Nanoparticles of the size of about 5 nm and larger can be clearly visualized by TEM. TEM is a useful method of detect the presence and subcellular localization of nanoparticles. TEM can also be used to estimate the density of nanoparticles in a region. A density calculation can be performed by counting the number of observed particles in a given area scanned by TEM. An understanding of the density of nanoparticles in a defined region, such as a cell's nucleus or cytoplasm, can provide information regarding the size requirements for a nanoparticle, the effectiveness of a given nuclear localization signal and other parameters.

Nanoparticles of the invention can also be detected spectroscopically. Ultraviolet (UV), visible and infrared (IR) spectroscopic methods can be employed. The choice of detection method will typically depend on the experimental design. In one embodiment, nanoparticles of the invention can be indirectly detected using fluorescence spectroscopy. Expression of GFP and other fluorescent marker proteins provided by the nanoparticles can be detected by fluorescence and can act as an indicator of the presence of a nanoparticles. A fluorescent moiety can be associated with the nanoparticle and the presence of the nanoparticle itself can be identified.

Microscopy techniques such as bright field microscopy, phase contrast microscopy, confocal microscopy and other techniques can be employed to detect the presence of nanoparticles. Phase contrast microscopy is typically used for the visualization of cellular organelles, and can be employed to detect the presence of nanoparticles. Confocal microscopy can also be useful for detecting nanoparticles. The resolution of any of the above microscopy techniques can be enhanced by the introduction of various contrast enhancement or other agents known to refine images and increase resolution.

Protein-based detection of a nanoparticle is also possible. For example, a labeled protein is bound to the nanoparticle. Alternatively, a first protein is attached to the nanoparticle. Then a second protein known to associate with a first protein bound to a nanoparticle can be labeled and used as a probe. Suitable labels include fluorescent moieties and other labels. Upon association of the first and second proteins, and therefore association of the labeled second protein on the nanoparticle, the presence of the nanoparticle is detectable by detecting the presence of the probe. Any suitable protein pair can be used to detect a nanoparticle of the invention.

### Kits and Devices

In another aspect, this invention provides a kit for contraception comprising, or alternatively consisting essentially of, or yet further consisting of: a plurality of positively charged nanoparticles; and instructions for use. In another aspect, this invention provides a kit for contraception comprising, or alternatively consisting essentially of, or yet further consisting of: a device containing a plurality of positively charged nanoparticles; and instructions for use. In some embodiments, there is provided a composition comprising an effective amount of the nanoparticle in a pharmaceutically acceptable carrier. In some embodiments, the pharmaceutically acceptable carrier in the kits is suitable for topical administration of the agent. The formulations can be for immediate or controlled release of the active ingredients. In some embodiments, the pharmaceutically acceptable carrier further comprises, or alternatively consists essentially of, or yet further consist of, a penetration or permeation enhancer.

Provided herein are kits for administration of the nanoparticles for inducing contraception, as described herein. Kits may further comprise suitable packaging and/or instructions for use of the compound. Kits may also comprise a means for the delivery of the composition and instructions for administration. Alternatively, the kit provides the compound and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, a tube or a container containing a topical composition of the nanoparticle composition, a vaginal ring, a tampon, a suppository, or an intrauterine device.

The kits may include other therapeutic compounds for use in conjunction with the compounds described herein. The other therapeutic compounds include, but are not limited to, conventional pain-killers, spermicides, etc. These compounds can be provided in a separate form or mixed with the compounds of the present invention.

The kits will include appropriate instructions for preparation and administration of the nanoparticles or the composition provided herein, side effects of the nanoparticles or the composition provided herein, and any other relevant information. The instructions can be in any suitable format, including, but not limited to, printed matter, videotape, computer readable disk, or optical disc.

In another aspect of the invention, kits for inducing contraception in a female mammal are provided, comprising a container containing the plurality of the positively charged nanoparticles or the composition described herein, and instructions for use. The container can be any of those known in the art and appropriate for storage and delivery of intravenous or topical formulations. Kits may also be provided that contain sufficient dosages of the effective composition or compound to provide effective treatment for an individual for an extended period, such as a week, 2 weeks, 3, weeks, 4 weeks, 6 weeks, or 8 weeks or more.

In one aspect, there is provided a device for contraception in a mammal, wherein the device comprises a means for administering an effective amount of a plurality of positively charged nanoparticles for aggregating mucus in a mammal, thereby causing contraception in the mammal. The means to administer an effective amount of the composition are selected from implants, tampon, suppository, vaginal rings, intrauterine device, diaphragm, condom, sponge, or just a tube containing a gel or paste of the composition of the invention. Such devices are well known to those in the art. Suitable devices for use in the present invention may be seen in, for example, in Remington: The Science and Practice of Pharmacy, 19th Edition, Chapter 95, pages 1676-1692, Mack Publishing Co., Easton, Pa. 1995. In some embodiments, the devices such as implants, tampon, suppository, vaginal rings, intrauterine device, diaphragm, condom, or sponge, contain or are coated with the compositions provided herein.

In some embodiments, the device is an implant, tampon, suppository, vaginal ring, intrauterine device, diaphragm, condom, or a sponge, composed of the composition comprising a plurality of the positively charged nanoparticles, optionally a spermicide such as Nonoxynol-9, and a water soluble polymer. In some embodiments, the amount of the spermicide used may vary from 3-30% based on the total weight of the device, or alternatively in the range of from 5-15% by weight. Based on the amount of the spermicide used in vaginal formulations and on the estimated concentration of the spermicide necessary to immobilize sperm in vaginal fluid, it is contemplated that devices release about 40 mg or more of the spermicide within the first hour and from 100-150 mg within 24 hours following insertion into the vagina.

In some embodiments, the water-soluble polymer is a waxy, low-molecular-weight compound that migrates to the surface of the device and provides lubrication on contact with the body. The waxes preferably should melt near or below body temperature to provide the desired lubrication. The incorporation of a water-soluble, low molecular weight polymer may also modify the release rate of the positively charged nanoparticle and optionally a spermicide from the device by providing additional pathways for diffusion as it is released. In some embodiments, the water soluble polymer is polyethylene glycol which has a molecular weight ranging from 600 to 6,000. The increased initial release of the composition from the subject diaphragm results in improved efficacy in the event of coitus immediately following insertion of the diaphragm.

In some embodiments, the range of the water soluble polymer incorporated into the composition is from 0 to 25% based on total weight of the device. Alternatively, the preferred range is about 10 to 15% by weight.

Unless otherwise stated all temperatures are in degrees Celsius. Also, in these examples and elsewhere, abbreviations have the following meanings

| | |
|---|---|
| HBSS = | Hanks' balanced salt solution |
| hrs = | hours |
| mM = | millimolar |
| mg/L = | milligram/liter |
| μm = | micrometer |
| nm = | nanometer |
| NP = | nanoparticle |

The following examples are provided to illustrate select embodiments of the invention as disclosed and claimed herein.

### EXAMPLES

### Example 1

### Effect of Nanoparticles (NPs) on the Aggregation of Mucin

### Materials:

In this study, the influence of the surface charge and size modifications of NPs on mucus rheology, was investigated. Further information about this example is found in Chen et al., (2010) PLoS One, 5(11):e15434, the content of which is incorporated into the present disclosure by reference in its entirety. Commercialized polystyrene NPs (Bangs Laboratory Inc, Fishers, Ind.) with dimensions of 57, 99, 120 and 160 nm were added to mucin samples. Positive (—NH₂ based, 57 and 160 nm), negative (—COOH, 120 nm) and neutral (hydrophobic, 99 nm) surface charges were used in this study. All NPs were prepared in suspension of Hanks' balanced salt solution (HBSS) containing 20 mM Tris-HCl (tris(hydroxymethyl)aminomethane hydrochloride) and 10 mM MES (2-(N-morpholino)ethanesulfonic acid) to buffer the pH around 7.3. Porcine gastric mucin from Sigma (St. Louis, Mo.) was dissolved in HBSS (Tris-HCIIMES buffering, pH 7.3) at 1 mg/L and filtered through a 0.22 μm membrane before adding different charged polystyrene NPs.

### Methods:

### A549 Cell Culture

The human lung carcinoma cell line A549 was obtained from American Type Culture Collection (ATCC, VA, USA). A549 cell line is an airway epithelial cell line commonly used as a secretory model (Berger J T, et al., (1999) Am J Respir Cell Mol Biol 20: 500-510). Cells were cultured in 15 cm cell culture plates (VWR, CA, USA) containing F-12 nutrient mixture medium (Invitrogen, CA, USA) that was supplemented with 100 U of penicillin/streptomycin and 10% heat-inactivated fetal bovine serum (FBS) (Invitrogen, CA, USA). The A549 lung cells were cultured in 15 cm Falcon plates and incubated in a humidified incubator at 37° C./5% CO₂. Cell counts were performed using trypan blue (Sigma-Aldrich, MO, USA) exclusion and a Bright-Line haemocytometer.

### Nanoparticle Preparation

Carboxyl-, amine- and non-functionalized polystyrene particles with various sizes (i.e. 57 nm, 99 nm, 120 nm and 160 nm) (Bangs Laboratories, Fishers, Ind., USA) were used in this study. All NPs have a size standard deviation of ≦10% (based on manufacturer information). These sizes were independently confirmed using homodyne dynamics laser scattering. All nanoparticle samples were sonicated before usage.

### Particle Sizing Using Dynamic Laser Scattering

The aggregation of mucus was monitored by measuring particle size using homodyne dynamics laser scattering (DLS). Samples of porcine gastric mucin at 1 mg/L (Sigma-Aldrich, MO, USA) were prepared with Hanks' solution containing 1.2 mM Ca²⁺, 20 mM Tris-HCl (tris(hydroxymethyl)aminomethane hydrochloride) and 10 mM MES (2-(N-morpholino)ethanesulfonic acid) (Sigma-Aldrich, MO, USA) to buffer the pH around 7.4 (Chen E Y et al., Am J Physiol Lung Cell Mol Physiol 299: L542-549). The solution was thoroughly mixed until mucin has dissolved. Aliquots of mucin samples (10 ml) were directly filtered through a 0.22-μm Millipore PES membrane (pre-washed with 0.1 N HCl) (Fisher Scientific, CA, USA) into clean scintillating vials. The scintillating vials were positioned in the goniometer of a Brookhaven laser spectrometer (Brookhaven Instruments, NY, USA). Mucin gel aggregation was allowed to take place by equilibrating with three filtered types of NPs each expressing different surface modifications for 72 hrs; they were subsequently analyzed by detecting the scattering fluctuations at a 45 degree scattering angle. Commercialized polystyrene NPs (Bangs Laboratories, Fishers, Ind.) with dimensions of 57, 99, 120 and 160 nm were added to mucin samples. Positive (—NH₂ based, 57 and 160 nm), negative (—COOH, 120 nm) and non-functionalized (hydrophobic, 99 nm) surface charges were used in this study. All NPs were prepared in suspension of Hanks' solution (buffered with Tris-HCl/MES at pH 7.4). Filtered NP solution, added to scintillating vials, was tested for its ability to promote mucin aggregation and was monitored at 1 hr, 3 hrs, 5 hrs, 24 hrs, 48 hrs and 72 hrs. Self aggregation induced by NP-only and mucin-only controls were monitored at 1 hr, 24 hrs, 48 hrs and 72 hrs. The pH was also monitored and maintained at approximately 7.4 during the experiments. The autocorrelation function of the scattering intensity fluctuations was averaged over a 3-min sampling time using a Brookhaven BI 9000AT autocorrelator. Particle size distribution was calculated by CONTIN (Chin W C et al., (1998) Nature 391: 568-572). Calibration was conducted with standard monodisperse suspensions of latex microspheres ranging from 50 nm to 10 μm (Polysciences, PA, USA).

### Scanning Electron Microscopy (SEM)

Samples of porcine gastric mucin at 1 mg/L (Sigma-Aldrich, MO, USA) were prepared with Hanks' solution (buffered with Tris-HCl/MES at pH 7.4) and incubated with 10 mg/L of positively-charged 57 and 160 nm NPs. After incubation for 5 hrs or 72 hrs, 5 ml of mucin were filtered through a 0.22-μm Millipore isopore membrane (Fisher Scientific, CA, USA). The filtered mucin was subsequently fixed with 4% paraformaldehyde (prepared in PBS at pH 7.4) (Sigma-Aldrich, MO, USA) for 5 min, followed by careful rinsing with Hanks' solution twice. The fixed mucin and NP complexes were dehydrated by soaking in serially diluted ethanol (35%, 50%, 70%, 95% and 100% ethanol) for 5 min. The specimens were air dried and examined with FEI Quanta 200 ESEM (North America NanoPort, OR, USA).

### EGTA Dispersion of Aggregated Mucin

EGTA (Ethylene glycol-bis(2-aminoethylether)-N,N,N′,N′-tetraaceticacid, 2 mM) (Sigma-Aldrich, MO, USA) was used to disperse aggregated mucus, which was monitored by measuring particle size by homodyne dynamics laser scattering. Samples of porcine gastric mucin at 1 mg/L (Sigma-Aldrich, MO, USA) were prepared with Hanks' solution containing 1.2 mM Ca²⁺ (buffered with Tris-HCl/MES at pH 7.4) and thoroughly mixed until mucin has dissolved. Aliquots of mucin samples (10 ml) were directly filtered through a 0.22-μm Millipore PES membrane (pre-washed with 0.1 N HCl) (Fisher Scientific, CA, USA) into clean scintillating vials. The scintillating vials were positioned in the goniometer of a Brookhaven laser spectrometer (Brookhaven Instruments, NY, USA). Mucin gel aggregation was initiated by incubating mucin samples with filtered positively-charged NPs (—NH₂ based 57 and 160 nm) (Bangs Laboratory Inc, Fishers, Ind.) and were monitored after 1, 3, 5, 24, 48 and 72 hrs. EGTA (2 mM) was subsequently applied to already aggregated mucin solution after 72 hrs. The change in aggregation size was detected for another 72 hrs. The pH was also monitored and maintained at approximately 7.4 during the experiments. The autocorrelation function of the scattering intensity fluctuations was averaged over a 3-min sampling time, using a Brookhaven BI 9000AT autocorrelator. Particle size distribution was calculated by the CONTIN (Chin W C et al., (1998) Nature 391: 568-572). Calibration was conducted with standard monodisperse suspensions of latex microspheres ranging from 50 nm to 10 μm (Polysciences Inc, PA, USA).

### Swelling Kinetics of Exocytosed Mucin Matrices

The A549 cell culture plates were rinsed with Hanks' buffer twice. Non-trypsin dissociation buffer (Invitrogen, CA, USA) was added to detach cells from the plates and were subsequently incubated at 37° C. for 15 minutes, centrifuged at 700 rpm for 5 min, and resuspended in Hanks' solution (Invitrogen, CA, USA) (buffered with Tris-HCl/MES at pH 7.4). Cells were resuspended into MatTek glass bottom dishes (MatTek Corporation, MA, USA) and equilibrated in a 37° C. incubator for 10 minutes prior to adding 10 mg/L and 1 mg/L of 160 and 57 nm positively-charged NPs (Bangs Laboratories, Fishers, Ind.). The pH was monitored and maintained at approximately 7.4 throughout the experiments.

A549 cells were viewed and video-recorded with phase-contrast lens using a Nikon Eclipse TE-2000-U inverted fluorescence microscope (Nikon Eclipse TE-2000U, Tokyo, Japan). Degranulation of A549 cells was induced by lᵢ μM ionomycin (Sigma-Aldrich, MO, USA) and was found to be a readily observable discrete quantal process. During exocytosis into extracellular Hanks' solution, released granule matrices undergo rapid swelling. Video-recordings of granular exocytosis and swelling were captured at 30 frames s⁻¹.

Measurements of radii, of the released mucin matrices, as a function of time were used to verify that the swelling of the secreted material followed the characteristic features of polymer gel swelling kinetics (Espinosa M et al., (2002) Hum Reprod 17: 1964-1972; Tanaka T and Fillmore D (1979) J Chem Phys 70: 1214-1218). The swelling of a polymer gel follows a typical diffusive kinetics that is independent of the size, internal topology, or chemical composition of the gel (Tanaka T and Fillmore D (1979) J Chem Phys 70: 1214-1218). For spherical gels as observed from the exocytosed mucin granule matrices of A549 cells, the radial dimension increased following a characteristic first order kinetics of the form r(t)=r_{f}−(r_{f}−rᵢ) e^{-t/τ} (Equation 1), where rᵢ and r_{f} are the initial and final radius of the secretory granule matrix, respectively, and τ is the characteristic relaxation time of the swelling process (Verdugo P (1990) Ann Rev Physiol 52: 157-176). The polymer network of gels diffused into the solvent (Hanks' solution), with a diffusivity (D) (D=(r_{f})²/τ[cm² s⁻¹]) (Equation 2). The diffusivity (D) of polyionic gels varied with the concentration of counterions in the swelling medium.

### Statistical Analysis

The data was presented as means±SD. Each experiment was performed independently at least three times. Statistical significance was determined using a Student's t-test analysis with p values of <0.005 (Microsoft Excel and GraphPad Prism 4.0, GraphPad Software, Inc., San Diego, Calif., USA).

### Results

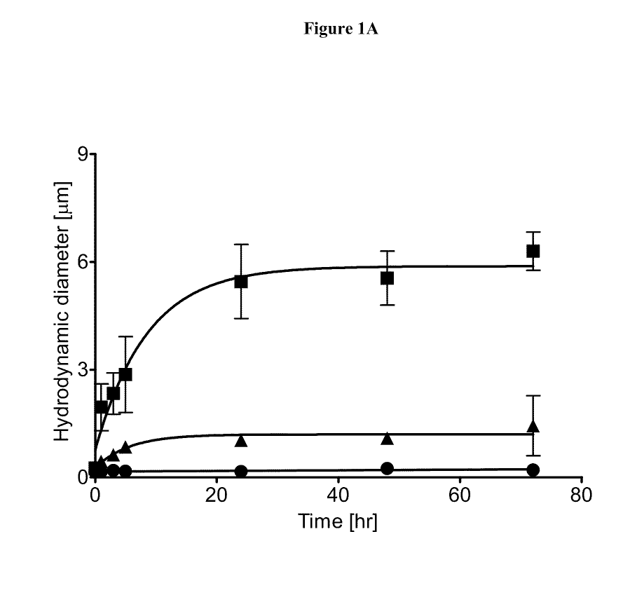

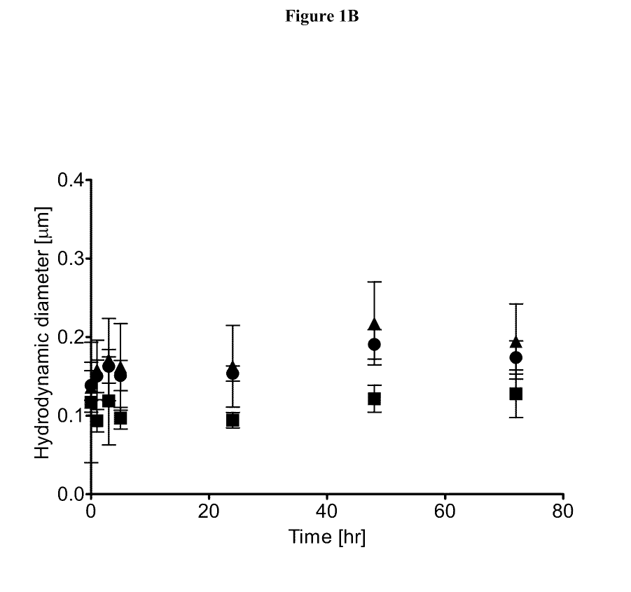

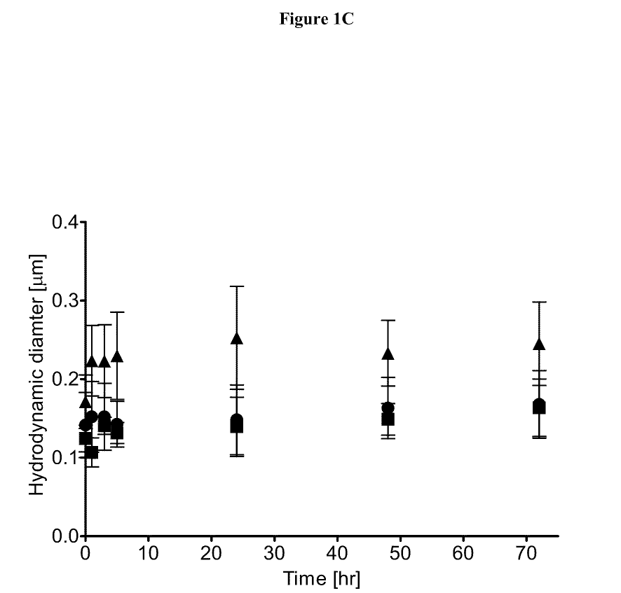

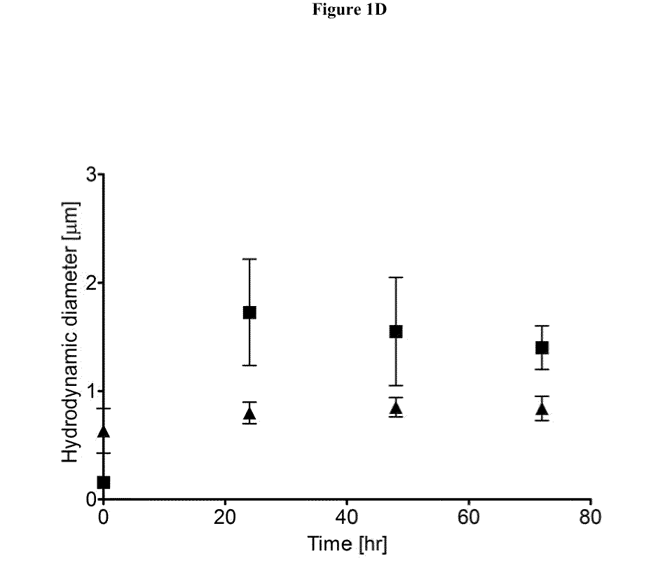

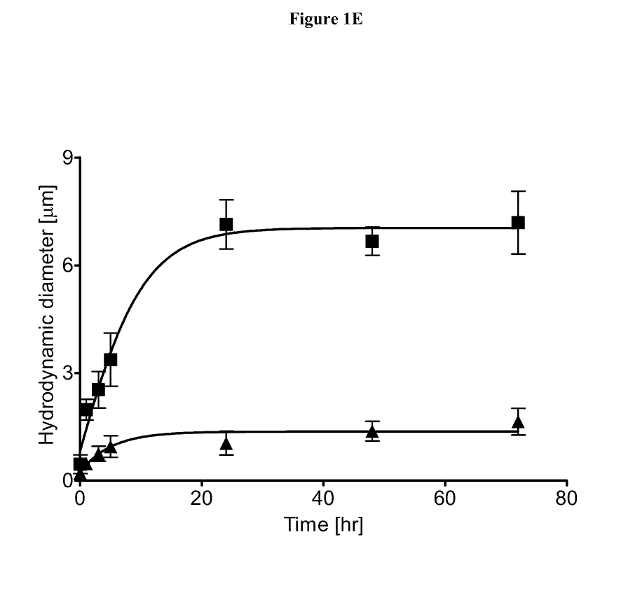

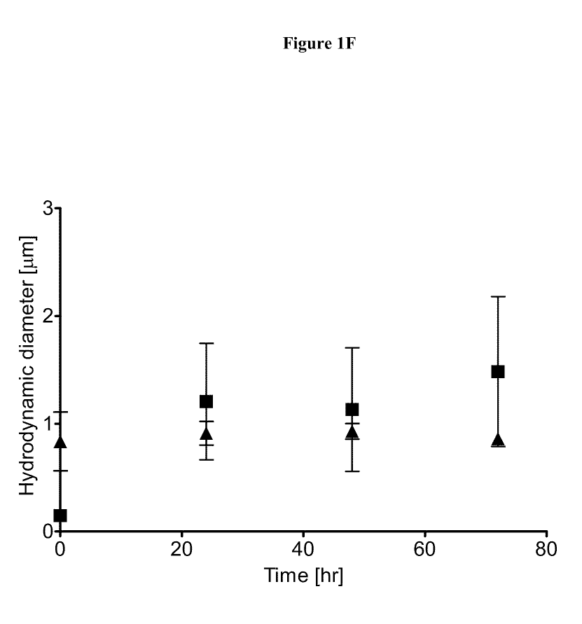

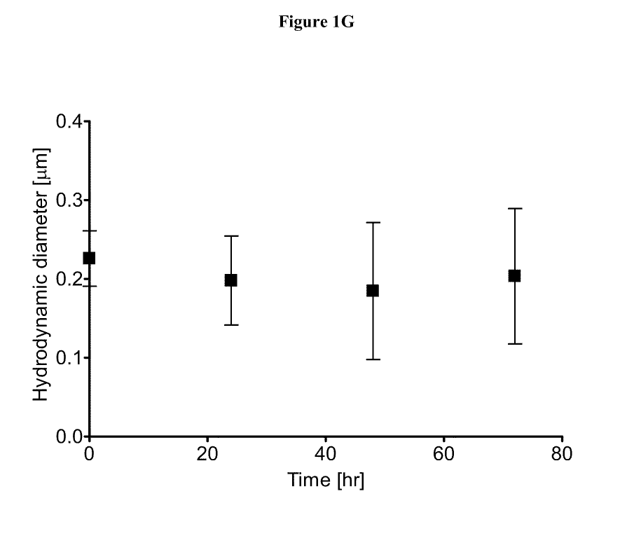

To examine the effects of NP surface properties on mucin aggregation and gelation, NPs with different surface charges were added to mucin samples to monitor the mucin gel size change with dynamic laser scattering (DLS) after 0, 1, 3, 5, 24, 48 and 72 hrs. DLS is a well established technique to measure the hydrodynamic diameter of mucin gel aggregates (Bhaskar et al. (1991) American Journal of Physiology 261(5):G827-G833). In FIG. 1A, it is clearly demonstrated that a steady concentration-dependent increase in mucin aggregate size was induced by positively-charged amine (—NH₂) polystyrene NPs (160 nm) at 1 mg/L and 10 mg/L. Positively-charged NPs (1 mg/L) caused mucin to aggregate from the original 240 nm to 1000 nm within 24 hrs and plateaued throughout 72 hrs. In comparison to the NP-only control (no mucin) at 1 mg/L, only minor aggregation was found throughout the 72 hrs (FIG. 1D). On the other hand, 10 mg/L of positively-charged NPs induced a quick rise in mucin aggregation starting from the 1st hour (˜2000 nm) to the 5th hour (˜2900 nm). At the 24th hour, the average mucin gel size increased rapidly to around 5500 nm while it plateaued around 72 hrs at 6300 nm (FIG. 1A). Comparing the treatment group to the mucin-only control, there is a 19 and 24 time increase in mucin aggregate size in the corresponding 24 and 72 hrs. However, at much lower concentrations, positively-charged NPs (100 μg/L) failed to induce significant mucin aggregation (FIG. 1A). In addition, multiple concentrations of negatively-(—COOH based, 120 nm) and neutral-charged (99 nm) NPs yielded a basal size of about 200 nm (FIGS. 1B & C).

Therefore, the data revealed the inability of negative and neutral NPs to promote mucin aggregation and mucus rheological changes (FIGS. 1B & C). It has been shown that hydrophobic and mucin-mucin interactions play vital roles in pH-induced gelation of gastric mucin (Cao et al. (1999) Biophys J 76(3):1250-8). However, the hydrophobic moieties on neutral NPs failed to promote significant mucin gelation in this study. This inability might be partially due to the low mucin concentration (1 mg/L) and the neutral pH (buffered at 7.3).

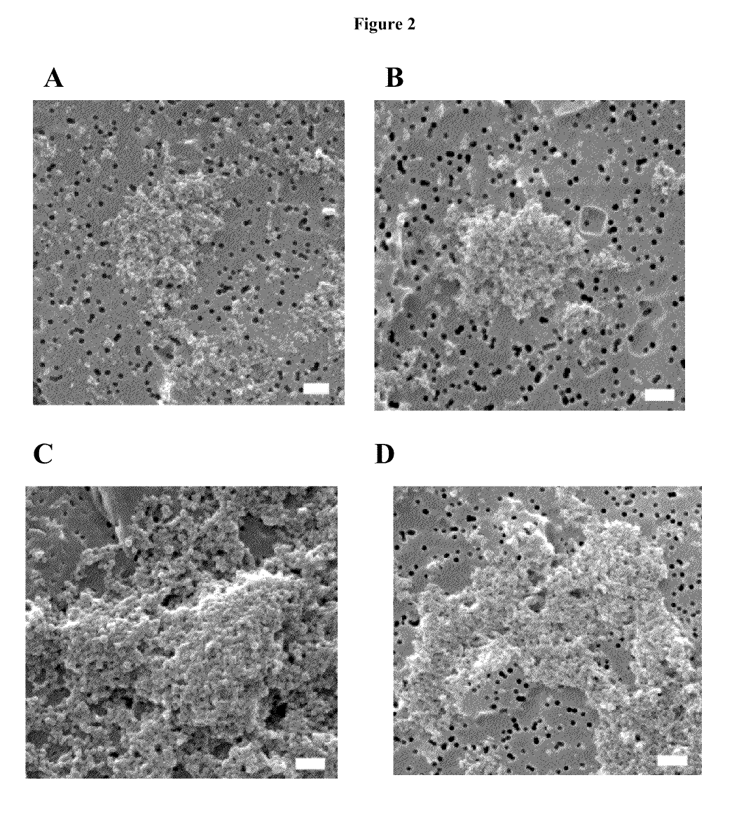

Moreover, FIG. 1E illustrated a NP size-dependent effect on mucin aggregation. Positively-charged 57 nm NPs generated more pronounced mucin aggregates than 160 nm positively charged NPs under the same conditions. The resultant larger mucin gels can be elucidated by differential charge density on NPs. Without being limited by any theory, inventors believe that smaller NPs have greater curvature which probably contain higher density of positively charged functionalized groups. The control experiment (FIGS. 1D & F) indicated that 10 mg/L of 160 and 57 nm positive charged NPs generated only slight self-clumping. Furthermore, 57 nm NP-induced mucin aggregates were approximately 30 times larger than the mucin-only control. The mucin-only control remained at the hydrodynamic diameter of about 150 nm throughout 72 hrs (FIG. 1G). These results suggested that NP-induced mucus aggregation occurred mainly as a result of interactions between NPs and mucin instead of self-aggregation of NPs or mucin. The data validated that NPs can induce mucin gelation, resulting in larger mucin aggregates (6-8 μm), compared with the sizes of low pH or temperature-induced mucus gelation. It is critical to point out that the resultant larger mucin aggregations are not purely mucin gelation but NP-mucin gel complexes, as can be clearly shown in the scanning electron microscopy (SEM) images (FIG. 2). The results suggest that NP-induced mucin aggregation (NP-mucin gel complex) takes place mainly via electrostatic interactions between positively-charged functionalized groups and anionic oligosacchalide side chains or carboxylate side groups on amino acids of mucin. Furthermore, the finding is corroborated by the fact that electrostatic interactions on mucin oligosaccharide side chains can control the gelation and viscoelastic properties of mucin.

The results show that NPs promote mucin to form large gels which become poorly dispersed and less transportable. To further establish the relationship between NP concentrations and the size of mucin gels, aggregated mucin size was determined by SEM images. Mucin samples containing 10 mg/L of 57 nm and 160 nm positively-charged NPs were prepared. The resulting mucin gels were collected at 5 hrs and 72 hrs. Mucin gels were filtered through 0.22 μm isopore membranes, fixed and imaged with SEM. Sizes of mucin aggregates presented in FIGS. 2A-D confirm the size measurements from DLS. It is also evident that longer incubation resulted in larger NP-mucin aggregates for 57 nm NPs (FIGS. 2B & D) and 160 nm NPs (FIGS. 2A & C), which is consistent with DLS measurements (FIGS. 1A & E). In addition, SEM images demonstrated that these aggregated mucin gels were associated with NPs forming NP-mucin gel complexes.

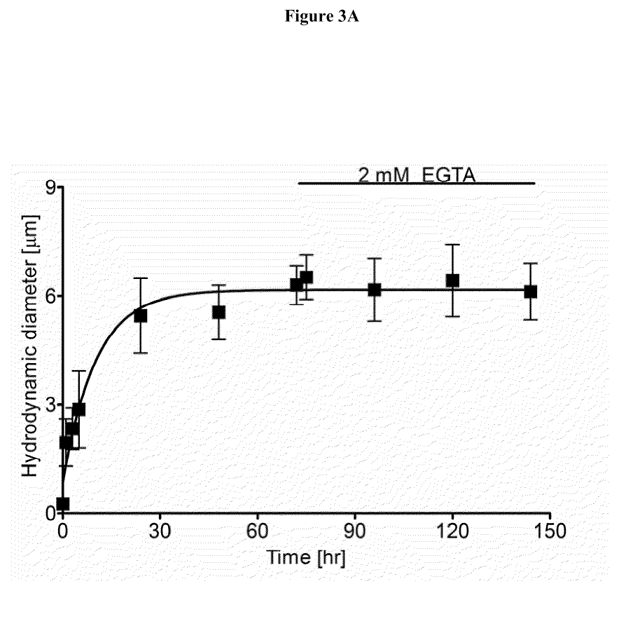

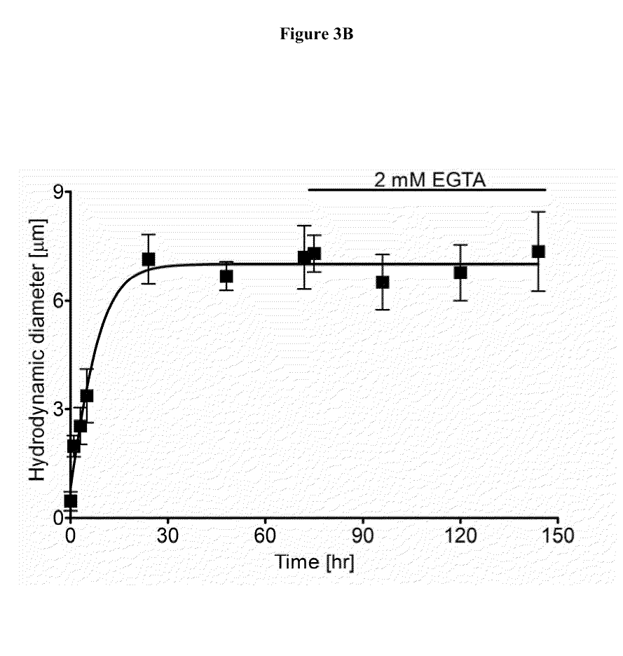

Calcium ions have been shown to act as a vital crosslinker in the formation and condensation of mucin gels (Verdugo (1990) Annu Rev. Physiol. 52:157-76). To further understand the role that positively-charged NPs play in promoting mucin aggregation and gelation, it was tested whether positive charged NPs can serve as crosslinker replacing calcium ions. FIG. 3 shows that the application of EGTA (Ca²⁺ chelator) failed to disperse aggregated mucin significantly. Positively-charged NPs (10 mg/L) of 160 nm and 57 nm were added to mucin solution (1 mg/L) and incubated for 72 hrs before applying 2 mM EGTA. The pH was kept in a range of pH 7.3 by Tris-HCl and MES. The decrease of aggregated mucin size was subsequently monitored after 3, 24, 48, and 72 hrs. FIGS. 3A & B demonstrated that calcium chelation by EGTA was unable to significantly reduce aggregated mucin size caused by 160 nm or 50 nm NPs. The previous results have shown that gels crosslinked by Ca²⁺ can be dispersed by Ca²⁺ chelators (e.g. EDTA) (Chin et al. (1998) Nature 391(6667):568-572). This result further reinforced the role of positive charged NPs as Ca²⁺ crosslinkers.

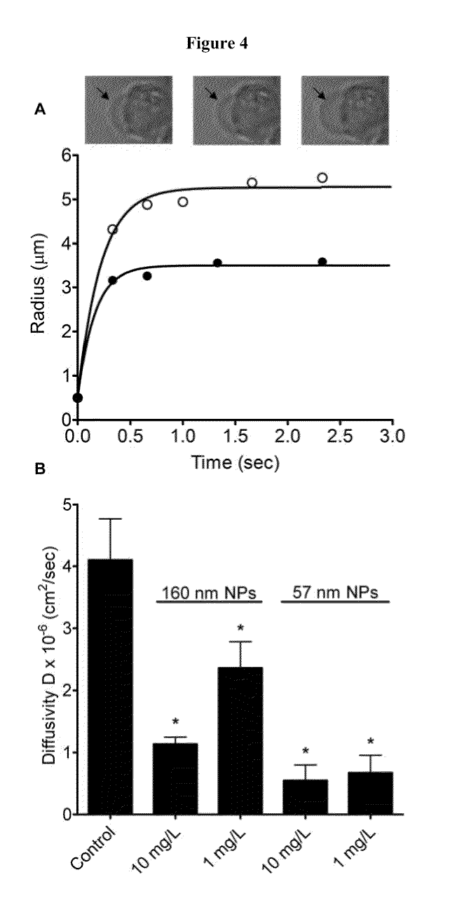

To confirm that the changes in rheological property of mucin can be induced by NPs, an in vitro functional assay involving mucin swelling kinetics has been examined. It is shown that positively-charged NPs (10 mg/L) of 160 and 57 nm retarded mucin diffusivity approximately 3.6 and 7.5 times, correspondingly (FIGS. 4A and 4B). The data indicates that positively-charged NPs can reduce mucin dispersion and hydration, possibly leading to viscous mucus and dysfunctional transport.

Mucus is mainly composed of large and heavily glycosylated glycoproteins called mucin. The gel-forming mucins rapidly hydrate after exocytosis and due to their tangle network properties, anneal with other mucins to form a protective barrier at the airway-surface liquid layer. The mucin gel layer lines the epithelial surface of various organs such as the vaginal tract, eyes, gastric wall and pulmonary lumen. Mucus in the airway of lungs serves as an innate immune defense against inhaled particulates, bacteria and viruses. Maintenance of the airway protection mechanism stems from the delicate balance between normal mucus production, transport and clearance. The mucin polymer network of mucus has a characteristic tangled topology. Since the rheological properties of mucus are governed mainly by the tangle density of mucin polymers, which decreases with the square of the volume of the mucin matrix, the mucin network hydration (degree of swelling) is the most critical factor in determining the rheological properties of mucus. The diffusivity of mucin matrices, which is closely related to mucin viscosity, can be calculated from polymer swelling kinetics. Based on the polymer network theory, polymer diffusivity is inversely proportional to its viscosity. Thus, lower rate of mucin diffusivity is associated with higher viscosity, less dispersed and less transportable mucins that appear to characterize the clinical symptoms of thick mucus accumulation and obstruction commonly found in asthma, COPD and CF. Although chronic exposure to NPs has been shown to induce excessive mucin synthesis and increase the risk of COPD, the connection between NPs and poorly dispersed viscous mucus has not been explored.

Due to the versatile application of NPs in nanocarrier delivery systems and commercial products, NPs with varying surface functionalization and characteristics are widely utilized. The polyanionic nature of mucin glycoproteins allows for possible electrostatic interactions with oppositely charged NPs. This study investigated how surface charges and size modifications of NPs influence mucus rheological properties. Mucus hydration can be significantly impeded by a change in electrolyte concentration in airway-surface liquid (ASL). Calcium ion concentration has been shown to increase from 1 mM to 4 mM in the ASL of CF patients, hindering proper mucus swelling and hydration and leading to respiratory health problems. So far there is no evidence documenting ASL abnormalities in NP-induced airway diseases. However, the mechanism by which NPs can cause changes in the rheological properties of mucus is still unknown.

Gastric mucin was used as a model mucin in the present investigation as it shares many similarities with airway mucins. The present approach is consistent with previous studies that used gastric mucin to examine the viscoelastic properties and biological applications of mucins, since there is currently no commercial mucin sample available that can be preserved in the native state. This study utilized DLS to probe the interactions between functionalized NPs and mucin. DLS is a common technique implemented to measure the Brownian motion of mucin polymer in solvent. One of the major advantages of this technique is that it enables mucin structure and dynamics to be examined in the native state without chemical fixation, which minimizes protein denaturation and artifacts. The diffusion constant that was calculated from Brownian motion measurement is inversely proportional to the hydrodynamic diameter of the measured particle; low diffusion constant usually correlates with large mucin gelation/aggregation. As demonstrated by FIGS. 1A, E & 2A-D, positively-charged NPs can promote mucin aggregation (NP-mucin gel complexes) in a concentration- and size-dependent manner. The mechanism may involve electrostatic attraction between positively-charged NPs and the polyanionic sites on mucin such as the sialic, sulfate and carboxyl functional groups promoting mucin aggregation. By the same notion, mucin polyanionic charges are neutralized by cationic calcium counterions which condenses the mucin matrix and facilitates gel formation. On the other hand, negatively-charged and non-functionalized NPs failed to generate significant mucin aggregation. Other studies have shown that hydrophobic and mucin-mucin interactions play vital roles in pH-induced gelation of gastric mucin. However, the hydrophobic moieties on non-functionalized NPs failed to promote significant mucin gelation in this study. This inability might be partially due to the low mucin concentration (1 mg/L) and the neutral pH (buffered at 7.4). The negatively-charged NPs are likely to repel anionic sialic, sulfate and carboxyl functional groups on mucin thereby hindering mucin aggregation. Another possible mechanism could be that these negatively-charged NPs can chelate divalent ions (e.g. Ca²⁺) in the solution, which play a critical role in mucin gel crosslinking.

In addition, these data validated that smaller NPs can induce the formation of larger size mucin aggregated gels more effectively (FIGS. 1E, 2B & D). The resulting larger mucin gels can possibly be elucidated by differential charge densities on NPs. These findings are supported by other studies showing that positively-charged chitosan coated NPs, or amine-modified NPs, were highly adhesive to polyanionic mucus gels. Viral particles with highly dense positive charges have also been proposed to be more mucoadhesive.

After establishing that positively-charged NPs can promote mucin gelation, the data suggests that positively-charged NPs may indeed act as network crosslinkers. FIGS. 3A & B confirmed that EGTA chelation of indigenous mucin network crosslinker Ca²⁺ ions was unable to disperse the NP-induced mucin aggregation/gelation. Therefore, positively-charged NPs crosslink the polyanionic tangle networks via electrostatic interactions. This evidence further validates the idea that positively-charged NPs effectively replace the natural role of Ca²⁺ ions and highlights the health danger with irreversibly strengthening the crosslinking within mucin gels.

To demonstrate the harmful consequence that positively-charged NPs can have through their crosslinking ability on mucus rheological properties, a swelling kinetics functional assay was used. The direct effect of positively-charged NPs was determined on the mucus hydration rate by measuring the swelling kinetics of newly exocytosed mucin matrices from human lung A549 cells under positively-charged NP exposure. A549 cells are a representative in vitro model system for studying mucin swelling kinetics as they express both major respiratory MUC SAC and MUC 5B mucin proteins. This study showed that positively-charged NPs hindered the rates of mucin matrix hydration and diffusivity in both a concentration- and size-dependent manner (FIGS. 4A & B). In accordance with the polymer network theory (lower diffusivity correlates with higher viscosity), the data indicated that positively-charged NPs decrease the rate of mucin diffusivity and can increase the viscosity of mucin network; the effect of which is further enhanced by smaller sized NPs. The experimental data has provided the first mechanistic link between positively-charged NPs and altered rheological properties of mucus.

The positively-charged NPs can crosslink mucins, thus forming NP-mucin gel complexes. The effects of which can potentially induce the formation of viscous mucus and hinder proper mucus hydration and dispersion, leading to impaired mucociliary transport in various epithelial mucosa. The outcome of viscous mucus accumulation could exacerbate pulmonary symptoms of diseased individuals and potentially elevate chances of morbidity in healthy pulmonary systems. As in the case of COPD, CF, or asthma patients, positively-charged NPs could worsen the problem by further thickening the mucus. The complications may include additional reduction in the airway flow or the promotion of chronic bacterial infection. This report also indicates some possible undesirable side effects of drug delivery using positively-charged nanocarriers via epithelial mucosa. These NPs might lead to drug entrapment within mucus, impedance of proper mucus dispersion, and transportability. This study also found that non-functionalized and negatively-charged NPs have less impact on the rheological properties of mucus. This finding provides the necessary knowledge needed for the safety consideration when using positively-charged functionalized NPs.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method to aggregate mucus, comprising contacting the mucus with an effective amount of a plurality of positively charged nanoparticles, thereby aggregating the mucus.

2. The method of claim 1, wherein the contacting causes a replacement of a calcium ion cross-linking a mucin in the mucus with the positively charged nanoparticles.

3. The method of claim 1, wherein the aggregation is irreversible.

4. The method of claim 1, wherein the contacting is performed in vitro or in vivo.

5. The method of claim 1, wherein the plurality of the effective amount of positively charged nanoparticles result in an increase in a size of the mucus to more than about 200 nm in less than about 1 hr from contacting or administration.

6. The method claim 1, wherein an average diameter of the plurality of the positively charged nanoparticles is less than about 500 nm.

7. The method of claim 1, wherein the average diameter of the plurality of the positively charged nanoparticle is between about 50 nm to about 200 nm.

8. The method of claim 1, wherein the positively charged nanoparticle comprises amine modified polystyrene, chitosan-sodium tripolyphosphate (TPP)-polyethylene glycol (PEG), polyethyleneimine (PEI), stearyl amine, 4-(dimethylamino) pyridine, and/or thiocholine.

9. A method of contraception in a female mammal, comprising: vaginally administering to the female mammal an effective amount of a plurality of positively charged nanoparticles, resulting in the contraception in the mammal.

10. The method of claim 10, wherein the effective amount causes an aggregation of the mucus in the female mammal.

11. A method to prevent fertilization of an egg by a sperm by aggregating cervical mucus in a female mammal, comprising: administering to the female mammal prior to insemination an effective amount of a plurality of positively charged nanoparticles to cause the aggregation of the cervical mucus in the female mammal, resulting in the prevention of the fertilization of the egg by the sperm in the female mammal.

12. The method of claim 9 or 11, wherein the nanoparticle is administered topically, via injection, diaphragm, tampon, suppository, condom, sponge, barrier, intrauterine device, or vaginal ring.

13. The method of claim 9 or 11, wherein the mammal is human, rat, mice, canine, leproid, bovine, ovine, feline, and equine.

14. The method of claim 9 or 11, further comprising administering an effective amount of a spermicide.

15. The method of claim 14, wherein the spermicide comprises detergent nonoxynol-9 (N-9, nonylphenoxypolyethoxyethanol), benzalkonium chloride, sodium cholate, p-diisobutylphenoxypolyethoxyethanol (octoxynol), p-methanylphenyl polyoxyethylene (8.8) ether (menfegol), dodecamethylene glycol monolaurate, sodium lauryl sulfate, or gramicidin.

16. A device for contraception in a mammal, wherein the device comprises a means for administering an effective amount of a plurality of positively charged nanoparticles for aggregating mucus in a mammal, thereby causing contraception in the mammal.

17. The device of claim 16, wherein the means to administer an effective amount of the composition is selected from the group consisting of a tube, ampule, injection, diaphragm, tampon, suppository, condom, sponge, barrier, intrauterine device, and vaginal ring.

18. An isolated complex of nanoparticle with mucin wherein the nanoparticle is positively charged before complexation with mucin.
